(19) United States
(12) Patent Application Publication
(10) Pub. No.: US 2014/0026899 A1
(43) Pub. Date: Jan. 30, 2014

(54) RESTRAINING STRAP DEVICE

(71) Applicants: James Giese, Marinette, WI (US); Loretta Giese, Marinette, WI (US)

(72) Inventors: James Giese, Marinette, WI (US); Loretta Giese, Marinette, WI (US)

(21) Appl. No.: 13/561,049

(22) Filed: Jul. 29, 2012

(51) Int. Cl. A61F 5/37

(52) U.S. Cl. 128/878

(57) ABSTRACT

The present invention is a restraining strap device that includes a wrist strap with a first end and a second end, the wrist snap has a snap fastener with a male portion and a female portion, the male portion of the snap fastener is disposed on the first end of the wrist strap and the female portion of the snap fastener is disposed on the second end of the wrist strap. The restraining strap device also includes an arm strap with a first end and a second end, the arm strap has a first loop and a second loop and the wrist strap is releasably attached to the arm strap.

### TECHNICAL FIELD & BACKGROUND

An individual with an irregular heartbeat may require a pacemaker to monitor their heart rate. Several weeks following surgery, many patients wear a sling to prevent disconnection of wires through heavy lifting and extreme movement of their arm, making it difficult to perform simple tasks.

The present invention generally relates to a strap. More specifically, the invention is a restraining strap device.

It is an object of the invention to provide a restraining strap device that is a medical accessory that supports a user's arm while allowing limited arm movement.

It is an object of the invention to provide a restraining strap device that allows relatively better arm movement for a user than a traditional sling.

What is really needed is a restraining strap device that is a medical accessory that supports a user's arm while allowing limited arm movement and that allows relatively better arm movement for a user than a traditional sling.

### BRIEF DESCRIPTION OF THE DRAWINGS

The present invention will be described by way of exemplary embodiments, but not limitations, illustrated in the accompanying drawings in which like references denote similar elements, and in which:

FIG. 1A illustrates a side view of a restraining strap device, according to an embodiment of the present invention.

FIG. 1B illustrates a side view of an assembled restraining strap device, according to an embodiment of the present invention.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention. However, the order of description should not be construed as to imply that these operations are necessarily order dependent. In particular, these operations need not be performed in the order of presentation.

The phrase “in one embodiment” is utilized repeatedly. The phrase generally does not refer to the same embodiment, however, it may. The terms “comprising”, “having” and “including” are synonymous, unless the context dictates otherwise.

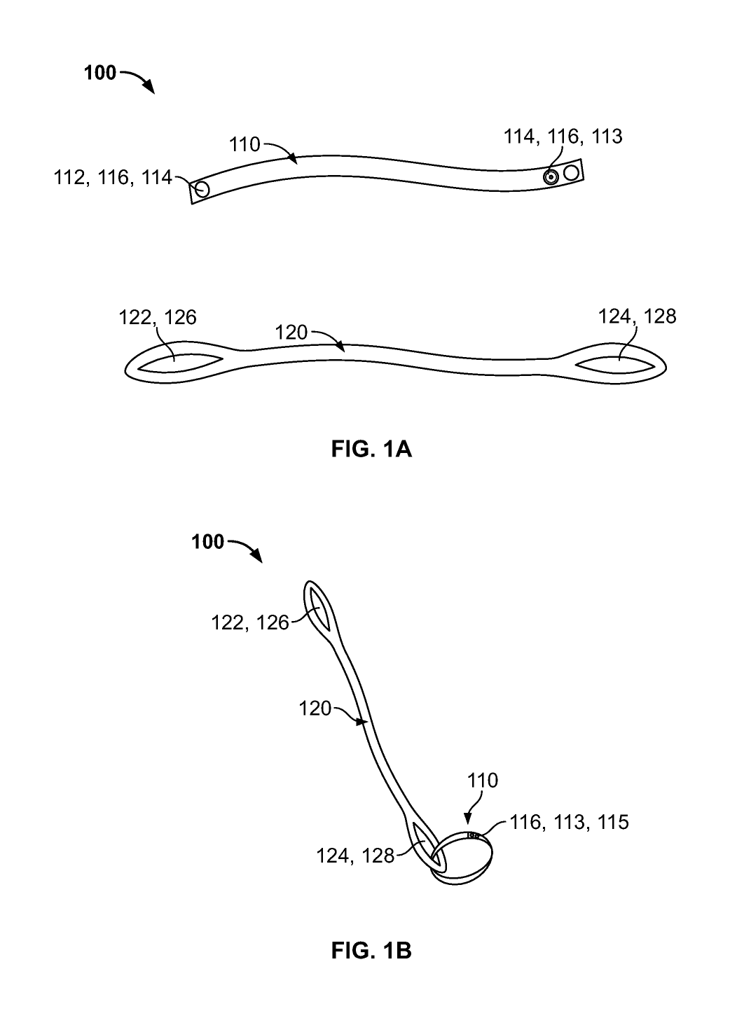

FIG. 1A illustrates a side view of a restraining strap device 100, according to an embodiment of the present invention. The restraining strap device 100 is a medical accessory that supports a user's arm (not shown) while allowing limited arm movement and that allows relatively better arm movement for a user than a traditional sling.

The restraining strap device 100 includes a wrist strap 110 and an arm strap 120. The wrist strap 110 has a first end 112 and a second end 114. The wrist snap 110 also has a snap fastener 116 with a male portion 113 and a female portion 115. The male portion 113 of the snap fastener 116 is disposed on the first end 112 of the wrist strap 110 and the female portion 115 of the snap fastener 116 is disposed on the second end 114 of the wrist strap 110, although any other suitable fastener such as a hook and loop fastener (not shown) can also be utilized. The wrist strap 110 is approximately 10 inches in length but can be any other suitable length. The arm strap 120 has a first end 122 and a second end 124. The arm strap 120 also has a first loop 126 and a second loop 128. The first loop 126 is disposed on the first end 122 of the arm strap 120 and the second loop 128 is disposed on the second loop 128 of the arm strap 120. The arm strap 120 is approximately 28 inches in length but can be any other suitable length. Additional details regarding the restraining strap device 100 is illustrated and described in subsequent FIG. 1B and its description.

FIG. 1B illustrates a side view of an assembled restraining strap device 100, according to an embodiment of the present invention.

The restraining strap device 100 illustrated in FIG. 1B has similar features to the restraining strap device 100 illustrated and described in FIG. 1A and its description that include a similar wrist strap 110 and an arm strap 120. The wrist strap 110 is secured around the second loop 128 of the arm strap 120 with the snap fastener 116 by releasably coupling the male portion 113 and the female portion 115 of the wrist strap 110 after inserting the wrist strap 110 through the second loop 128 of the arm strap 120. The first loop 126 of the arm strap 120 goes around a user's wrist (not shown) to provide support to the user's arm while allowing limited movement of the user's arm. The wrist strap 120 that is inserted through the second loop 128 of the arm strap 120 is wrapped around a belt loop or a pant loop (not shown) of the user to secure the wrist strap 120 to the user.

The restraining strap device allows relatively moderate mobility of a user's arm. The restraining strap device is a wrist and arm strap that goes from a user's belt or pant loop to the user's wrist. The restraining strap device may be relatively quickly and easily adjusted to provide optimal comfort for users. Patients who have undergone minor surgery may find the restraining strap device both convenient and easy to use. The restraining strap device features a device that limits a user's range of motion, but provides mobility for an injured arm. The restraining strap device is designed with an adjustable wrist strap for use by a wide variety of patient users.

While the present invention has been related in terms of the foregoing embodiments, those skilled in the art will recognize that the invention is not limited to the embodiments described. The present invention can be practiced with modification and alteration within the spirit and scope of the appended claims. Thus, the description is to be regarded as illustrative instead of restrictive on the present invention.

1. A restraining strap device, comprising:
a wrist strap with a first end and a second end, said wrist snap has a snap fastener with a male portion and a female portion; and
an arm strap with a first end and a second end, said arm strap has a first loop and a second loop, said wrist strap is releasably attached to said arm strap.

2. The restraining strap device according to claim 1, wherein said male portion of said snap fastener is disposed on said first end of said wrist strap.

3. The restraining strap device according to claim 1, wherein said female portion of said snap fastener is disposed on said second end of said wrist strap.

4. The restraining strap device according to claim 1, wherein said first loop is disposed on said first end of said arm strap.

5. The restraining strap device according to claim 1, wherein said first loop of said arm strap goes around a user's wrist to provide support to a user's arm while allowing limited movement of said user's arm.

6. The restraining strap device according to claim 1, wherein said second loop is disposed on said second end of said arm strap.

7. The restraining strap device according to claim 1, wherein said wrist strap is secured around said second loop of said arm strap with said snap fastener by releasably coupling said male portion and said female portion of said wrist strap.

8. The restraining strap device according to claim 7, wherein said wrist strap is secured around said second loop of said arm strap after inserting said wrist strap through said second loop of said arm strap.

9. The restraining strap device according to claim 8, wherein said wrist strap that is inserted through said second loop of said arm strap, said second loop is wrapped around a belt loop of said user to secure said wrist strap to said user.

10. The restraining strap device according to claim 8, wherein said wrist strap that is inserted through said second loop of said arm strap, said second loop is wrapped around a pant loop of a user to secure said wrist strap to said user.

11. A restraining strap device, comprising:
a wrist strap with a first end and a second end, said wrist snap has a snap fastener with a male portion and a female portion, said male portion of said snap fastener is disposed on said first end of said wrist strap, said female portion of said snap fastener is disposed on said second end of said wrist strap; and
an arm strap with a first end and a second end, said arm strap has a first loop and a second loop, said wrist strap is releasably attached to said arm strap.

12. The restraining strap device according to claim 11, wherein said first loop is disposed on said first end of said arm strap.

13. The restraining strap device according to claim 11, wherein said first loop of said arm strap goes around a user's wrist to provide support to a user's arm while allowing limited movement of said user's arm.

14. The restraining strap device according to claim 11, wherein said second loop is disposed on said second end of said arm strap.

15. The restraining strap device according to claim 11, wherein said wrist strap is secured around said second loop of said arm strap with said snap fastener by releasably coupling said male portion and said female portion of said wrist strap.

16. The restraining strap device according to claim 15, wherein said wrist strap is secured around said second loop of said arm strap after inserting said wrist strap through said second loop of said arm strap.

17. The restraining strap device according to claim 16, wherein said wrist strap that is inserted through said second loop of said arm strap, said second loop is wrapped around a belt loop of said user to secure said wrist strap to said user.

18. The restraining strap device according to claim 16, wherein said wrist strap that is inserted through said second loop of said arm strap, said second loop is wrapped around a pant loop of a user to secure said wrist strap to said user.

19. The restraining strap device according to claim 11, wherein said wrist strap is approximately 10 inches in length.

20. The restraining strap device according to claim 11, wherein said arm strap is approximately 28 inches in length.
